(19) United States
(12) Patent Application Publication
(10) Pub. No.: US 2026/0041719 A1
(43) Pub. Date: Feb. 12, 2026

(54) GUT MICROBIOMES AND ASSESSING AND TREATING CANCER

(71) Applicant: Mayo Foundation for Medical Education and Research, Rochester, MN (US)

(72) Inventors: Nabila Nora Bennani, Rochester, MN (US); Thomas E. Witzig, Rochester, MN (US); Purna C. Kashyap, Rochester, MN (US)

(21) Appl. No.: 19/144,064

(22) PCT Filed: Oct. 27, 2023
(86) PCT No.: PCT/US2023/036099

§ 371 Date: Jun. 27, 2025

Related U.S. Application Data
(60) Provisional application No. 63/435,919, filed on Dec. 29, 2022.

(51) Int. Cl. A61K 35/741; A61P 35/00; C12Q 1/06

(52) U.S. Cl. CPC A61K 35/741; A61P 35/00; C12Q 1/06

(57) ABSTRACT

This document relates to methods and materials involved in assessing and/or treating a mammal having cancer (e.g., lymphoma). For example, methods and materials that can be used to determine if a mammal (e.g., a human) having cancer (e.g., lymphoma) is likely to respond to or has a cancer or a gut microbiome that makes that mammal more responsive to a particular cancer treatment are provided. Methods and materials for treating a mammal having cancer (e.g., lymphoma) are also provided.

### CROSS-REFERENCE TO RELATED APPLICATIONS

This application claims priority from U.S. Provisional Application Ser. No. 63/435,919, filed Dec. 29, 2022. The disclosure of the prior application is considered part of (and is incorporated by reference in) the disclosure of this application.

### STATEMENT REGARDING FEDERAL FUNDING

This invention was made with government support under CA097274 awarded by the National Institutes of Health. The government has certain rights in the invention.

### TECHNICAL FIELD

This document relates to methods and materials involved in assessing and/or treating a mammal having cancer (e.g., lymphoma). For example, methods and materials provided herein can be used to determine if a mammal (e.g., a human) having cancer (e.g., lymphoma) is likely to respond to or has a cancer or a gut microbiome that makes that mammal more responsive to a particular cancer treatment. Methods and materials provided herein can be used to aid in the selection of a cancer treatment for a mammal having cancer (e.g., lymphoma) based on an analysis of that mammal's gut microbiome.

This document also provides methods and materials for treating a mammal having cancer (e.g., lymphoma). For example, methods and materials provided herein can be used to treat a mammal with cancer (e.g., lymphoma) by using particular cancer treatments that have an effect on or perturb that mammal's gut microbiome independently from or in combination with other cancer treatments.

### BACKGROUND INFORMATION

The gut microbiome has been implicated as a factor in cancer pathogenesis, response to treatment, and the development of side effects of treatment. The gut microbiome plays a role in immune response to disease by affecting, among other things, the inflammatory response, the response to infection, and tolerance for food and other commensal or environmental antigens. The gut microbiome is connected with cancer treatment outcomes and toxicities to different cancer treatments in different cancer patient populations (Helmink et al., Nat. Med., 25:377-388 (2019)). Among cancers, lymphoma, for instance, can involve the gastrointestinal tract, which is a location for different types of immune cells (Yu et al., BMC Cancer, 21: 934 (2021); and Routy et al., Science, 359(6371):91-97 (2018)). The microorganisms (or microbes) present in a mammal's gut microbiome can influence or be influenced by whether a mammal has cancer, such as lymphoma.

### SUMMARY

This document provides methods and materials for assessing and/or treating mammals (e.g., humans) having cancer (e.g., lymphoma). For example, the total number of microbes identified and/or the quantity of some or all of the microbes identified in a mammal's gut microbiome can be used to determine if the mammal having cancer is likely to respond to or has a cancer that is more responsive to a particular cancer treatment or if a particular cancer treatment is preferred. In some cases, a gut microbiome of a mammal having cancer (e.g., lymphoma) can be used to determine if that mammal is likely to respond to a particular cancer treatment or is more susceptible to that cancer. For example, a sample (e.g., a stool sample) obtained from a mammal having cancer (e.g., lymphoma) can be assessed to determine if the mammal is likely to respond to a particular cancer treatment based, at least in part, on the gut microbiome of the sample. In some cases, a sample obtained from a mammal having cancer (e.g., lymphoma) can be assessed to aid in the selection of a preferred cancer treatment based, at least in part, on the gut microbiome identified using that sample.

As described herein, mammals (e.g., humans) having cancer (e.g., lymphoma) can have a lower diversity of microorganisms in their gut microbiome compared to controls (e.g., household controls) regardless of the gut involvement of that cancer. Also as described herein, mammals (e.g., humans) having cancer (e.g., lymphoma) and an elevated level of Fusobacteria as compared to controls (e.g., household controls) can be identified as being likely to have a poor outcome or a shorter cancer free survival than a comparable mammal not having an elevated level of Fusobacteria. In some cases, a mammal (e.g., a human) having cancer (e.g., lymphoma) and an elevated level of Fusobacteria as compared to controls (e.g., household controls) can be identified as being in need of a treatment that reduces the level of Fusobacteria within the mammal and/or a treatment that increases the level of Akkermansia and/or Barnesiellaceae within the mammal. In addition, as described herein, mammals (e.g., humans) having cancer (e.g., lymphoma) and a reduced level of Akkermansia and/or Barnesiellaceae as compared to controls (e.g., household controls) can be identified as being likely to have a poor outcome or as being likely to have a shorter cancer free survival than a comparable mammal not having reduced level of Akkermansia and/or Barnesiellaceae. In some cases, a mammal (e.g., a human) having cancer (e.g., lymphoma) and a reduced level of Akkermansia and/or Barnesiellaceae as compared to controls (e.g., household controls) can be identified as being in need of a treatment that increases the level of Akkermansia and/or Barnesiellaceae within the mammal and/or a treatment that reduces the level of Fusobacteria within the mammal.

In some cases, a mammal (e.g., a human) having cancer (e.g., lymphoma) and determined to have an elevated level of Fusobacteria as compared to controls (e.g., household controls) can be identified as being likely to have a poor outcome or a shorter cancer free survival than a comparable mammal not having an elevated level of Fusobacteria. In some cases, a mammal (e.g., a human) having cancer (e.g., lymphoma) and determined to have a reduced level of Fusobacteria as compared to controls (e.g., household controls) can be identified as being unlikely to have a poor outcome or as being likely to have a longer cancer free survival than a comparable mammal having an elevated level of Fusobacteria.

In some cases, a mammal (e.g., a human) having cancer (e.g., lymphoma) and determined to have a reduced level of Akkermansia and/or Barnesiellaceae as compared to controls (e.g., household controls) can be identified as being likely to have a poor outcome or a shorter cancer free survival than a comparable mammal not having the reduced level of Akkermansia and/or Barnesiellaceae. In some cases, a mammal (e.g., a human) having cancer (e.g., lymphoma) and determined to have an elevated level of Akkermansia and/or Barnesiellaceae as compared to controls (e.g., household controls) can be identified as being unlikely to have a poor outcome or as being likely to have a longer cancer free survival than a comparable mammal having a reduced level of Akkermansia and/or Barnesiellaceae.

This document also provides methods and materials for treating a mammal having cancer (e.g., lymphoma). For example, a mammal (e.g., a human) having cancer (e.g., lymphoma) that was identified as having an elevated level of Fusobacteria as compared to controls (e.g., household controls) can be administered one or more agents that reduce the level of Fusobacteria within the mammal (e.g., one or more antibiotics) and/or one or more agents that increase the level of Akkermansia and/or Barnesiellaceae within the mammal (e.g., one or more probiotics, synbiotics, or compositions containing viable Akkermansia and/or Barnesiellaceae). In another example, a mammal (e.g., a human) having cancer (e.g., lymphoma) that was identified as having a reduced level of Akkermansia and/or Barnesiellaceae as compared to controls (e.g., household controls) can be administered one or more agents that increase the level of Akkermansia and/or Barnesiellaceae within the mammal (e.g., one or more probiotics, synbiotics, or compositions containing viable Akkermansia and/or Barnesiellaceae) and/or one or more agents that reduce the level of Fusobacteria within the mammal (e.g., one or more antibiotics).

Having the ability to assess and/or treat mammals having cancer as described herein can allow clinicians and patients to achieve longer cancer-free survival times for the patients.

In general, one aspect of this document features methods for classifying a mammal having cancer as being likely to have a shortened cancer-free survival time. The methods can include, or consist essentially of, (a) identifying a mammal having cancer as having the presence of (i) an elevated level of Fusobacteria within the mammal's gut microbiome, (ii) a reduced level of Akkermansia within the mammal's gut microbiome, or (iii) a reduced level of Barnesiellaceae within the mammal's gut microbiome, and (b) classifying the mammal as being likely to have the shortened cancer-free survival time. The mammal can be a human. The cancer can be a lymphoma. The method can include identifying the mammal as having the presence of the elevated level of Fusobacteria. The elevated level of Fusobacteria can be greater than about 0.1% of the microbes present in the gut microbiome. The method can include identifying the mammal as having the presence of the reduced level of Akkermansia. The reduced level of Akkermansia can be less than about 1% of the microbes present in the gut microbiome. The method can include identifying the mammal as having the presence of the reduced level of Barnesiellaceae. The reduced level of Barnesiellaceae can be less than about 1% of the microbes present in the gut microbiome. The shortened cancer-free survival time can be as compared to the cancer-free survival time of comparable mammals lacking the presence of (i), (ii), and (iii).

In another aspect, this document features methods for classifying a mammal having cancer as being unlikely to have a shortened cancer-free survival time. The methods can include, or consist essentially of, (a) identifying a mammal having cancer as having the absence of (i) an elevated level of Fusobacteria within the mammal's gut microbiome, (ii) a reduced level of Akkermansia within the mammal's gut microbiome, and (iii) a reduced level of Barnesiellaceae within the mammal's gut microbiome, and (b) classifying the mammal as being unlikely to have the shortened cancer-free survival time. The mammal can be a human. The cancer can be a lymphoma. The elevated level of Fusobacteria can be greater than about 0.1% of the microbes present in the gut microbiome. The reduced level of Akkermansia can be less than about 1% of the microbes present in the gut microbiome. The reduced level of Barnesiellaceae can be less than about 1% of the microbes present in the gut microbiome. The shortened cancer-free survival time can be as compared to the cancer-free survival of comparable mammals having the presence of any one or more of (i), (ii), and (iii).

In another aspect, this document features methods for classifying a mammal having cancer as being likely to have a longer cancer-free survival time. The methods can include, or consist essentially of, (a) identifying a mammal having cancer as having the absence of (i) an elevated level of Fusobacteria within the mammal's gut microbiome, (ii) a reduced level of Akkermansia within the mammal's gut microbiome, and (iii) a reduced level of Barnesiellaceae within the mammal's gut microbiome, and (b) classifying the mammal as being likely to have the longer cancer-free survival time. The mammal can be a human. The cancer can be a lymphoma. The elevated level of Fusobacteria can be greater than about 0.1% of the microbes present in the gut microbiome. The reduced level of Akkermansia can be less than about 1% of the microbes present in the gut microbiome. The reduced level of Barnesiellaceae can be less than about 1% of the microbes present in the gut microbiome. The longer cancer-free survival time can be as compared to the cancer-free survival time of comparable mammals having the presence of any one or more of (i), (ii), and (iii).

In another aspect, this document features methods for classifying a mammal having cancer as being unlikely to have a longer cancer-free survival time. The methods can include, or consist essentially of, (a) identifying a mammal having cancer as having the presence of (i) an elevated level of Fusobacteria within the mammal's gut microbiome, (ii) a reduced level of Akkermansia within the mammal's gut microbiome, or (iii) a reduced level of Barnesiellaceae within the mammal's gut microbiome, and (b) classifying the mammal as being unlikely to have the longer cancer-free survival time. The mammal can be a human. The cancer can be a lymphoma. The method can include identifying the mammal as having the presence of the elevated level of Fusobacteria. The elevated level of Fusobacteria can be greater than about 0.1% of the microbes present in the gut microbiome. The method can include identifying the mammal as having the presence of the reduced level of Akkermansia. The reduced level of Akkermansia can be less than about 1% of the microbes present in the gut microbiome. The method can include identifying the mammal as having the presence of the reduced level of Barnesiellaceae. The reduced level of Barnesiellaceae is less than about 1% of the microbes present in the gut microbiome. The longer cancer-free survival time can be as compared to the cancer-free survival of comparable mammals lacking the presence of (i), (ii), and (iii).

In another aspect, this document features methods for treating a mammal having cancer. The methods can include, or consist essentially of, (a) identifying a mammal having cancer as having a reduced level of Akkermansia within the mammal's gut microbiome, and (b) administering to the mammal a composition comprising viable Akkermansia, where the method is effective to improve survival of the mammal. The mammal can be a human. The cancer can be a lymphoma. The reduced level of Akkermansia can be less than about 1% of the microbes present in the gut microbiome. The Akkermansia can include A. muciniphila, A. muciniphilia, A. glycaniphila, or any combinations thereof. The Akkermansia can be deposited with the Agricultural Research Service (ARS) Patent Culture Collection under NRRL number B-68146. The Akkermansia can be deposited with the ARS Patent Culture Collection under NRRL number B-68147. The method can include administering to the mammal a lymphoma treatment.

In another aspect, this document features methods for treating a mammal having cancer and identified as having a reduced level of Akkermansia within the mammal's gut microbiome. The methods can include, or consist essentially of, administering to a mammal having cancer and identified as having a reduced level of Akkermansia within the mammal's gut microbiome a composition comprising viable Akkermansia, where the method is effective to improve survival of the mammal. The mammal can be a human. The cancer can be a lymphoma. The reduced level of Akkermansia can be less than about 1% of the microbes present in the gut microbiome. The Akkermansia can incude A. muciniphila, A. muciniphilia, A. glycaniphila, or any combinations thereof. The Akkermansia can be deposited with the ARS Patent Culture Collection under NRRL number B-68146. The Akkermansia can be deposited with the ARS Patent Culture Collection under NRRL number B-68147. The method can include administering to the mammal a lymphoma treatment.

In another aspect, this document features methods for treating a mammal having cancer. The methods can include, or consist essentially of, (a) identifying a mammal having cancer as having a reduced level of Barnesiellaceae within the mammal's gut microbiome, and (b) administering to the mammal a composition comprising viable Barnesiellaceae, where the method is effective to improve survival of the mammal. The mammal can be a human. The cancer can be a lymphoma. The reduced level of Barnesiellaceae can be less than about 1% of the microbes present in the gut microbiome. The Barnesiellaceae can include B. viscericola, B. intestinihominis, B. sp904502265, B. excrementigallinarum, B. excrementipullorum, B. excrementavium, B. merdipullorum, B. merdigallinarum, or any combinations thereof. The Barnesiellaceae can be deposited with the ARS Patent Culture Collection under NRRL number B-68145. The method can include administering to the mammal a lymphoma treatment.

In another aspect, this document features methods for treating a mammal having cancer and identified as having a reduced level of Barnesiellaceae within the mammal's gut microbiome. The methods can include, or consist essentially of, administering to a mammal having cancer and identified as having a reduced level of Barnesiellaceae within the mammal's gut microbiome a composition comprising viable Barnesiellaceae, where the method is effective to improve survival of the mammal. The mammal can be a human. The cancer can be a lymphoma. The reduced level of Barnesiellaceae can be less than about 1% of the microbes present in the gut microbiome. The Barnesielaceae can include B. viscericola, B. intestinihominis, B. sp904502265, B. excrementigallinarum, B. excrementipullorum, B. excrementavium, B. merdipullorum, B. merdigallinarum, or any combinations thereof. The Barnesiellaceae can be deposited with the ARS Patent Culture Collection under NRRL number B-68145. The method can include administering to the mammal a lymphoma treatment.

In another aspect, this document features methods for treating a mammal having cancer. The methods can include, or consist essentially of, administering to a mammal having cancer a composition comprising viable Akkermansia or viable Barnesiellaceae, where the method is effective to improve survival of the mammal. The mammal can be a human. The cancer can be a lymphoma. The composition can include viable Akkermansia and viable Barnesiellaceae. The viable Akkermansia can include A. muciniphila, A. muciniphilia, A. glycaniphila, or any combinations thereof. The Akkermansia can be a Akkermansia of NRRL number B-68146 of the ARS Patent Culture Collection. The Akkermansia can be a Akkermansia of NRRL number B-68147 of the ARS Patent Culture Collection. The viable Barnesiellaceae can include B. viscericola, B. intestinihominis, B. sp904502265, B. excrementigallinarum, B. excrementipullorum, B. excrementavium, B. merdipullorum, B. merdigallinarum, or any combinations thereof. The Barnesiellaceae can be a Barnesiellaceae of NRRL number B-68145 of the ARS Patent Culture Collection. The method can include administering to the mammal a lymphoma treatment.

In another aspect, this document features methods for treating a mammal having cancer. The methods can include, or consist essentially of, (a) identifying a mammal having cancer as having an elevated level of Fusobacteria within the mammal's gut microbiome, and (b) administering to the mammal a composition comprising an antibiotic to reduce the level of the Fusobacteria within the mammal, where the method is effective to improve survival of the mammal. The mammal can be a human. The cancer can be lymphoma. The elevated level of Fusobacteria can be greater than about 0.1% of the microbes present in the gut microbiome. The Fusobacteria can be E. nucleatum, F. polymorphum, F. ulcerans, E. varium, F. mortiferum, F. perfoetens, E. animalis, F. vincentii, E. awasookii, F. periodonticum, F. russii, F necrophorum, F. gonidiaformans, or any combinations thereof. The method can include administering to the mammal a lymphoma treatment.

In another aspect, this document features methods for treating a mammal having cancer and identified as having an elevated level of Fusobacteria within the mammal's gut microbiome. The methods can include, or consist essentially of, administering to a mammal having cancer and identified as having an elevated level of Fusobacteria within the mammal's gut microbiome a composition comprising an antibiotic to reduce the level of the Fusobacteria within the mammal, where the method is effective to improve survival of the mammal. The mammal can be a human. The cancer can be lymphoma. The elevated level of Fusobacteria can be greater than about 0.1% of the microbes present in the gut microbiome. The Fusobacteria can be F. nucleatum, F. polymorphum, E. ulcerans, E. varium, E. mortiferum, F. perfoetens, F. animalis, F. vincentii, E. awasookii, F. periodonticum, F. russii, E. necrophorum, E. gonidiaformans, or any combinations thereof. The method can include administering to the mammal a lymphoma treatment.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A and 1B are schematic representations of the methods. FIG. 1A is diagram of the study design. Stool samples were collected from patients with untreated lymphoma prior to starting therapy; controls were stool samples from a human living in the same household (household controls). FIG. 1B is a diagram of the data analysis. Gut involvement refers to patients with cancer (lymphoma) involvement of the gastrointestinal (GI) tract as documented by endoscopy or tumor imaging. This could refer to the stomach or the small or large intestine. The samples were analyzed with the variables being α and β-diversity scores and taxa abundance. These measurements were first compared between patients and their household controls (where available). This was followed by analysis of the 3 variables with clinical characteristics and outcome measures such as event-free (EFS) and overall survival (OS). The statistical methods used in the analyses include Linear Mixed Models (LMM), generalized linear mixed model (GLMM), Cox proportional hazards model (Cox PH), and Microbiome Regression-Based Kernel Association Test (MiRKAT and MiRKAT-S).

FIG. 2 is a plot showing that the microbial content of the stool of patients is more similar to that of a paired household control than to the unpaired cases (P<0.001). The y-axis shows the Bray-Curtis (BC) distance between subjects. BC distance is a measure of dissimilarity of the microbial content.

FIG. 3 is a graph showing microbial alpha diversity (Shannon index) in lymphoma patients. Alpha diversity relates to the breadth of taxa represented within the sample. Lymphoma patients have a lower more restricted microbial diversity compared to controls (overall P=0.001). Lymphoma patients with GI tract lymphoma have a lower microbial alpha diversity than lymphoma patients without known GI tract involvement.

FIG. 4 contains the principal coordinate plot showing microbial community structure in lymphoma patients with or without GI tract involvement (“Yes”—with GI involvement, “No”—without GI involvement), compared to the household controls (P=0.002). The principal coordinate plot is based on the weighted UniFrac distance, and the first two principal coordinates (PC1 and PC2) capture 16.1% and 6.9% of the overall microbiome variability.

FIG. 5 contains the cladograms showing the change in relative abundance of specific taxa in lymphoma patients, compared to the household controls. “GI Yes”: lymphoma patients with GI tract involvement, “GI No”: lymphoma patients without GI tract involvement, “GI Yes&No”: all lymphoma patients. The cladogram depicts the hierarchical structure of the taxonomic groups tested. Highlighted nodes represent taxa with false discovery rate (FDR)<=0.05.

FIG. 6 contains graphs plotting the proportion of Akkermansia. “Yes” refers to lymphoma patients having involvement of the GI tract with lymphoma; “No” refers to lymphoma patients without GI tract involvement; and “Control” refers to household controls. Lymphoma patients have lower levels of the beneficial Akkermansia.

FIG. 7 contains graphs plotting the proportion of Fusobacteria. “Yes” refers to lymphoma patients having involvement of the GI tract; “No” refers to lymphoma patients without involvement of the GI tract; and “Control” refers to household controls. The GI tract involvement patients have higher levels of Fusobacteria.

FIG. 8 contains a plot showing event free survival (EFS) of diffuse large B cell lymphoma (DLBCL) patients. An event is defined as being refractory to initial therapy, relapsed after responding to initial therapy, or death due to any cause. The stool microbiome analysis results were divided by alpha diversity scores (Shannon index) into Low Diversity Index and High Diversity Index. Diversity index low refers to Shannon index less than or equal to the median. Diversity index high refers to Shannon index greater than the median. Patients with stools that had a narrower alpha diversity score (less number of taxa) were more likely to have an event (P=0.046).

FIG. 9 contains a graph showing EFS of lymphoma patients by levels of Akkermansia (FDR-adjusted p-value <0.1). Those patients having elevated levels of Akkermansia genus had a superior EFS compared to patients having the same disease but having stool with lower levels of Akkermansia. Akkermansia group low refers to less than or equal to the median, and Akkermansia group high refers to greater than the median.

FIG. 10 is a graph showing EFS of all lymphoma patients having elevated levels of Barnesiellaceae (FDR-adjusted p-value <0.1). Patients having elevated levels of Barnesiellaceae had superior EFS compared to those patients having the same disease but having stool with lower levels of these species. Barnesiellaceae group low refers to less than or equal to the median, and Barnesiellaceae group high refers to less than or equal to the median.

### DETAILED DESCRIPTION

This document provides methods and materials for assessing and/or treating mammals (e.g., humans) having cancer (e.g., lymphoma). For example, methods and materials provided herein can be used to determine if a mammal having cancer is likely to respond to a particular cancer treatment. In some cases, a gut microbiome of a mammal having cancer can be used to determine if that mammal is likely to respond to a particular cancer treatment. For example, a sample (e.g., a stool sample) obtained from a mammal having cancer can be assessed to determine if the mammal is likely to respond to a particular cancer treatment based, at least in part, on the gut microbiome of the sample. As described herein, a distinct gut microbiome can be present in a mammal (e.g., a human) having cancer (e.g., lymphoma) that is likely to respond to a particular cancer treatment. In some cases, an elevated level of Fusobacteria in a gut microbiome of a sample obtained from a mammal (e.g., a human) having cancer (e.g., lymphoma) can be used to identify that mammal as being likely to respond to a treatment that reduces the level of Fusobacteria within the mammal (e.g., one or more antibiotics). In some cases, a reduced level of Akkermansia and/or Barnesiellaceae in a gut microbiome of a sample (e.g., a stool sample) obtained from a mammal (e.g., a human) having cancer (e.g., lymphoma) can be used to identify that mammal as being likely to respond to a treatment that increases the level of Akkermansia and/or Barnesiellaceae within the mammal (e.g., one or more probiotics or compositions containing viable Akkermansia and/or Barnesiellaceae).

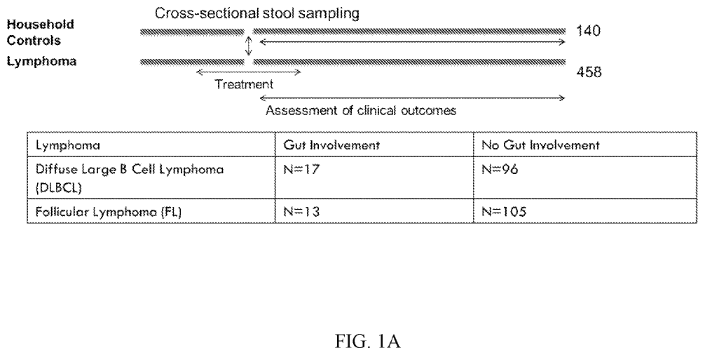

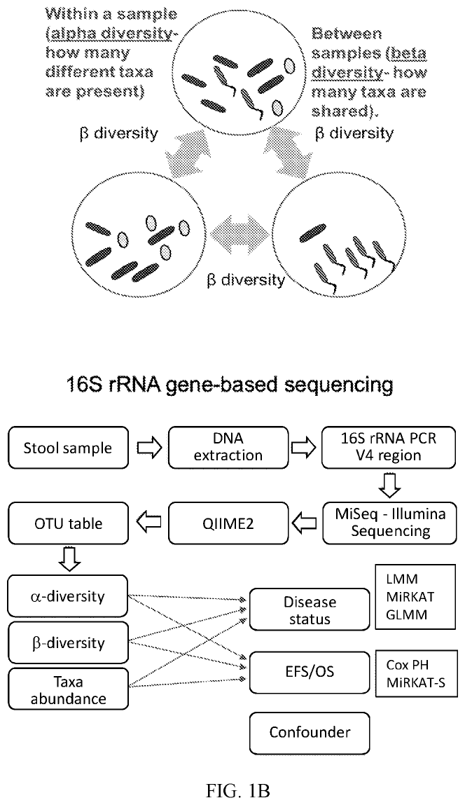

The term “elevated level” as used herein with respect to a level of a Fusobacteria refers to any level that is greater than a reference level of the Fusobacteria. The term “reference level” as used herein with respect to a Fusobacteria refers to the level of the Fusobacteria typically observed in a sample (e.g., a control sample) from one or more mammals (e.g., humans) without cancer (e.g., lymphoma). A Fusobacteria can be any species of Fusobacteria. Examples of species of Fusobacteria include, without limitation, F. nucleatum, F. polymorphum, F. ulcerans, F. varium, F. mortiferum, F. perfoetens, F. animalis, F. vincentii, F. awasookii, F. periodonticum, F. russii, F. necrophorum, and F. gonidiaformans. In some cases, a Fusobacteria can be as described elsewhere (see, e.g., Yeoh et al., Gut, 69(11):1998-2007, at, for example, FIGS. 1-4). Control samples can include, without limitation, samples (e.g., stool samples) from normal (e.g., healthy) mammals. In some cases, a control sample can be obtained from mammal that eats a similar diet and/or lives in a similar geographic location. For example, a control sample can be obtained from a mammal that lives in the same home (e.g., a household control). In some cases, an elevated level of Fusobacteria can be a level that is at least 2 (e.g., at least 5, at least 10, at least 15, at least 20, at least 25, at least 35, or at least 50) fold greater than a reference level of the Fusobacteria. In some cases, an elevated level of Fusobacteria can be a level that is greater than 200 Fusobacteria per 100,000 total microbes present in a sample (e.g., a gut microbiome sample). For example, an elevated level of Fusobacteria can be a level that is from about 200 Fusobacteria per 100,000 total microbes to about 30000 Fusobacteria per 100,000 total microbes (e.g., from about 200 to about 20000 Fusobacteria per 100,000 total microbes, from about 200 to about 10000 Fusobacteria per 100,000 total microbes, from about 200 to about 5000 Fusobacteria per 100,000 total microbes, from about 200 to about 2500 Fusobacteria per 100,000 total microbes, from about 200 to about 1000 Fusobacteria per 100,000 total microbes, from about 200 to about 500 Fusobacteria per 100,000 total microbes, from about 500 to about 30000 Fusobacteria per 100,000 total microbes, from about 750 to about 30000 Fusobacteria per 100,000 total microbes, from about 1000 to about 30000 Fusobacteria per 100,000 total microbes, from about 5000 to about 30000 Fusobacteria per 100,000 total microbes, from about 8000 to about 30000 Fusobacteria per 100,000 total microbes, from about 10000 to about 30000 Fusobacteria per 100,000 total microbes, from about 20000 to about 30000 Fusobacteria per 100,000 total microbes, from about 500 to about 20000 Fusobacteria per 100,000 total microbes, from about 1000 to about 10000 Fusobacteria per 100,000 total microbes, from about 2500 to about 5000 Fusobacteria per 100,000 total microbes, from about 500 to about 5000 Fusobacteria per 100,000 total microbes, from about 1000 to about 10000 Fusobacteria per 100,000 total microbes, or from about 5000 to about 20000 Fusobacteria per 100,000 total microbes). In some cases, an elevated level of Fusobacteria can be a level that is greater than 0.2% of the microbes present in the gut microbiome. For example, an elevated level of Fusobacteria can be a level that is from about 0.2% of the microbes present in the gut microbiome to about 30% of the microbes present in the gut microbiome. In some cases, when control samples have undetectable levels of a Fusobacteria, an elevated level can be a detectable level of the Fusobacteria. It will be appreciated that levels from comparable samples are used when determining whether or not a particular level is an elevated level.

The term “reduced level” as used herein with respect to a level of an Akkermansia or a Barnesiellaceae refers to any level that is less than a reference level of the Akkermansia or the Barnesiellaceae. The term “reference level” as used herein with respect to an Akkermansia or a Barnesiellaceae refers to the level of the Akkermansia or the Barnesiellaceae typically observed in a sample (e.g., a control sample) from one or more mammals (e.g., humans) without cancer (e.g., lymphoma). An Akkermansia can be any species of Akkermansia. Examples of species of Akkermansia include, without limitation, A. muciniphila, A. muciniphilia, and A. glycaniphila. A Barnesiellaceae can be any species of Barnesiellaceae. Examples of species of Barnesiellaceae include, without limitation, B. viscericola, B. intestinihominis, B. sp904502265, B. excrementigallinarum, B. excrementipullorum, B. excrementavium, B. merdipullorum, and B. merdigallinarum. Control samples can include, without limitation, samples (e.g., stool samples) from normal (e.g., healthy) mammals. In some cases, a control sample can be obtained from mammal that eats a similar diet and/or lives in a similar geographic location. For example, a control sample can be obtained from a mammal that lives in the same home (e.g., a household control). In some cases, a reduced level of an Akkermansia or Barnesiellaceae can be a level that is at least 2 (e.g., at least 5, at least 10, at least 15, at least 20, at least 25, at least 35, or at least 50) fold less than a reference level of the Akkermansia or the Barnesiellaceae. In some cases, a reduced level of Akkermansia can be a level that is less than 1000 Akkermansia per 100,000 total microbes (e.g., about 900 Akkermansia per 100,000 total microbes, about 800 Akkermansia per 100,000 total microbes, about 700 Akkermansia per 100,000 total microbes, about 600 Akkermansia per 100,000 total microbes, about 500 Akkermansia per 100,000 total microbes, about 400 Akkermansia per 100,000 total microbes, about 300 Akkermansia per 100,000 total microbes, about 200 Akkermansia per 100,000 total microbes, about 100 Akkermansia per 100,000 total microbes, or less than about 100 Akkermansia per 100,000 total microbes) present in a sample (e.g., a gut microbiome sample). For example, a reduced level of Akkermansia can be a level that is from about 0 Akkermansia per 100,000 total microbes to about 1000 Akkermansia per 100,000 total microbes (e.g., from about 0 to about 800 Akkermansia per 100,000 total microbes, from about 0 to about 500 Akkermansia per 100,000 total microbes, from about 0 to about 300 Akkermansia per 100,000 total microbes, from about 0 to about 200 Akkermansia per 100,000 total microbes, from about 0 to about 100 Akkermansia per 100,000 total microbes, from about 0 to about 50 Akkermansia per 100,000 total microbes, from about 50 to about 1000 Akkermansia per 100,000 total microbes, from about 100 to about 1000 Akkermansia per 100,000 total microbes, from about 200 to about 1000 Akkermansia per 100,000 total microbes, from about 500 to about 1000 Akkermansia per 100,000 total microbes, from about 800 to about 1000 Akkermansia per 100,000 total microbes, from about 50 to about 800 Akkermansia per 100,000 total microbes, from about 100 to about 600 Akkermansia per 100,000 total microbes, from about 200 to about 500 Akkermansia per 100,000 total microbes, from about 50 to about 100 Akkermansia per 100,000 total microbes, from about 100 to about 500 Akkermansia per 100,000 total microbes, or from about 500 to about 800 Akkermansia per 100,000 total microbes). In some cases, a reduced level of Akkermansia can be a level that is less than 1% of the microbes present in the gut microbiome. In some cases, a reduced level of Barnesielaceae can be a level that is less than 100 Barnesiellaceae per 100,000 total microbes present in a sample (e.g., a gut microbiome sample). For example, a reduced level of Barnesiellaceae can be a level that is from about 0 Barnesiellaceae per 100,000 total microbes to about 100 Barnesiellaceae per 100,000 total microbes (e.g., from about 0 to about 80 Barnesiellaceae per 100,000 total microbes, from about 0 to about 50 Barnesiellaceae per 100,000 total microbes, from about 0 to about 30 Barnesiellaceae per 100,000 total microbes, from about 10 to about 100 Barnesiellaceae per 100,000 total microbes, from about 30 to about 100 Barnesiellaceae per 100,000 total microbes, from about 50 to about 100 Barnesiellaceae per 100,000 total microbes, from about 70 to about 100 Barnesiellaceae per 100,000 total microbes, from about 10 to about 80 Barnesiellaceae per 100,000 total microbes, from about 20 to about 50 Barnesiellaceae per 100,000 total microbes, from about 10 to about 30 Barnesiellaceae per 100,000 total microbes, from about 30 to about 50 Barnesiellaceae per 100,000 total microbes, or from about 50 to about 80 Barnesiellaceae per 100,000 total microbes). In some cases, a reduced level of Barnesielaceae can be a level that is less than 1% of the microbes present in the gut microbiome. It will be appreciated that levels from comparable samples are used when determining whether or not a particular level is a reduced level.

Any appropriate mammal (e.g., a mammal having cancer such as a lymphoma) can be assessed and/or treated as described herein. Examples of mammals that can have cancer (e.g., lymphoma) and can be assessed and/or treated as described herein include, without limitation, humans, non-human primates (e.g., monkeys), dogs, cats, horses, cows, pigs, sheep, mice, and rats. In some cases, a mammal (e.g., a human) having cancer can have one or more gastrointestinal (GI) symptoms. Examples of GI symptoms that be experienced by a mammal having cancer include, without limitation, abnormal GI tract motility, abnormal GI tract sensation, and brain-gut GI tract dysfunction. In some cases, the mammal can be a human. For example, a human having cancer (e.g., lymphoma) can be assessed and/or treated as described herein. In some cases, a mammal (e.g., a human) identified as being immunosuppressed (e.g., having been administered one or more immunosuppressive therapies such as immune checkpoint inhibitors and cellular therapies) can be assessed and/or treated as described herein. For example, a mammal (e.g., a human) that is post organ transplant can be assessed and/or treated as described herein. In another example, a mammal (e.g., a human) having one or more autoimmune conditions (e.g., primary biliary cirrhosis and rheumatologic conditions such as rheumatoid arthritis and lupus (e.g., systemic lupus erythematosus)) can be assessed and/or treated as described herein. In a further example, a mammal (e.g., a human) having one or more gastrointestinal diseases (e.g., inflammatory bowel disease, and ulcerative colitis, Crohn's disease) can be assessed and/or treated as described herein. In some cases, a mammal (e.g., a human) having one or more non-malignant conditions (e.g., one or more organ transplants, one or more autoimmune conditions, and/or one or more gastrointestinal diseases) and being administered or having been administered one or more chemotherapies to treat the non-malignant condition(s) can be assessed and/or treated as described herein.

Any appropriate sample can be assessed to determine a gut microbiome of a mammal (e.g., a human) having cancer (e.g., lymphoma). In some cases, a sample can be a biological sample. A sample can be a fresh sample or a frozen sample. For example, biological samples such as stool samples, fluid samples (e.g., saliva samples), tissue samples (e.g., gingiva samples, breast tissue samples, vaginal tissue sample, and uterine tissue samples), can be obtained from a mammal and assessed to determine the gut microbiome of the mammal. In some cases, microbes (e.g., bacteria) can be isolated from a sample. For example, microbes (e.g., bacteria) can be isolated from a sample obtained from a mammal (e.g., a human) and can be used to determine a gut microbiome of the mammal. In some cases, one or more biological molecules can be isolated from microbes within a sample. For example, nucleic acid (e.g., RNA such as ribosomal RNA (rRNA)) can be isolated from microbes within a sample obtained from a mammal (e.g., a human) and can be used to determine a gut microbiome of the mammal. In some cases, microbes can be isolated from a stool sample obtained from a mammal (e.g., a human) having cancer (e.g., lymphoma) and can be assessed to determine a gut microbiome of the mammal.

Any appropriate method can be used to detect the presence, absence, or level of one or more microbes (e.g., Fitsobacteria, Akkermansia, and/or Barnesiellaceae) within a sample (e.g., a stool sample) obtained from a mammal (e.g., a human) having cancer (e.g., lymphoma). In some cases, sequencing techniques (e.g., 16S rRNA-sequencing and next-generation sequencing), shotgun metagenomics, quantitative PCR, and/or DNA hybridization-based techniques such as fluorescence in situ hybridization (FISH) can be used to identify the presence, absence, or level of a microbe within a sample (e.g., a stool sample) obtained from a mammal and to determine the gut microbiome of the mammal. In some cases, the presence, absence, or level of one or more microbes (e.g., Fusobacteria, Akkermansia, and/or Barnesiellaceae) within a sample (e.g., a stool sample) obtained from a mammal (e.g., a human) having cancer (e.g., lymphoma) can be determined as described in Example 1.

When assessing and/or treating a mammal (e.g., a human) having cancer as described herein, the cancer can be any type of cancer. In some cases, a cancer can be a blood cancer (e.g., lymphomas and leukemias). In some cases, a cancer can include one or more solid tumors. In some cases, a cancer can be a primary cancer. In some cases, a cancer can be a metastatic cancer. Examples of cancers that can be treated as described herein include, without limitation, lymphomas (e.g., Hodgkin's lymphomas and non-Hodgkin's lymphomas), breast cancers, lung cancers (e.g., non-small cell lung cancers), prostate cancers, esophageal cancers, pancreatic cancers, bladder cancers, melanoma, kidney cancers, brain cancers, bile duct cancers, gastric cancers, hepatobiliary cancers, rectal cancers, ovarian cancers, colon cancers, connective and soft tissue cancers, endometrial cancers, cervical cancers, oropharynx cancers, liver cancers, anal cancers, skin cancers, gallbladder cancers, bone cancers, head and neck cancers, myelomas, Waldenstroms macroglobulinemias, uterine cancers, and sarcomas.

In some cases, the methods described herein can include identifying a mammal (e.g., a human) as having cancer (e.g., lymphoma). Any appropriate method can be used to identify a mammal as having cancer. For example, physical examination, laboratory testing (e.g., blood tests), imaging techniques (e.g., ultrasound, magnetic resonance imaging (MRI), bone scan, computerized tomography (CT) scan, and positron emission tomography (PET) scan), and biopsy techniques can be used to identify a mammal (e.g., a human) as having cancer.

In some cases, the methods and materials provided herein can be used to determine an outcome (e.g., to predict treatment response) of a mammal (e.g., a human) having cancer (e.g., lymphoma). For example, the particular gut microbiome (e.g., the presence, absence, or level of one or more microbes such as Fusobacteria, Akkermansia, and/or Barnesiellaceae) of a mammal (e.g., a human) having cancer (e.g., lymphoma) can be used to determine if that mammal is likely to respond to a particular cancer treatment or is more susceptible to that cancer. In some cases, the presence, absence, or level of one or more microbes such as Fusobacteria, Akkermansia, and/or Barnesiellaceae within a sample (e.g., a stool sample) obtained from a mammal (e.g., a human) having cancer (e.g., lymphoma) can be used to determine if that mammal is likely to respond to a particular cancer treatment or is more susceptible to that cancer.

In some cases, the presence, absence, or level of one or more microbes (e.g., Fusobacteria, Akkermansia, and/or Barnesiellaceae) in a gut microbiome of a mammal (e.g., a human) having cancer (e.g., lymphoma) and/or identified as being immunosuppressed can be used to predict quality of life of the mammal. In some cases, a mammal (e.g., a human) having cancer (e.g., lymphoma) and/or identified as being immunosuppressed can be identified as being likely to experience a reduced quality of life based, at least in part, on the presence of an elevated level of Fusobacteria, a reduced level of Akkermansia, and/or a reduced level of Barnesiellaceae in the mammal's gut microbiome. For example, a mammal (e.g., a human) having cancer (e.g., lymphoma) and/or identified as being immunosuppressed, and also having an elevated level of Fusobacteria, a reduced level of Akkermansia, and/or a reduced level of Barnesiellaceae in a sample (e.g., a stool sample) obtained from the mammal can be identified as being likely to experience a reduced quality of life. In some cases, a mammal (e.g., a human) having cancer (e.g., lymphoma) and/or identified as being immunosuppressed can be identified as being likely to experience a better quality of life based, at least in part, on the absence of an elevated level of Fusobacteria, the absence of a reduced level of Akkermansia, and/or the absence of a reduced level of Barnesiellaceae in the mammal's gut microbiome. For example, a mammal (e.g., a human) having cancer (e.g., lymphoma) and/or identified as being immunosuppressed, and also lacking an elevated level of Fusobacteria, a reduced level of Akkermansia, and/or a reduced level of Barnesiellaceae in a sample (e.g., a stool sample) obtained from the mammal can be identified as being likely to experience a better quality of life. A quality of life can be assessed based on, for example, fatigue, infection, inflammation, changes in appetite, effects on sleep, and/or discomfort (e.g., due to altered bowel habits such as constipation and/or diarrhea).

In some cases, the presence, absence, or level of one or more microbes (e.g., Fusobacteria, Akkermansia, and/or Barnesiellaceae) in a gut microbiome of a mammal (e.g., a human) having cancer (e.g., lymphoma) can be used to predict the mammal's risk of relapse. In some cases, a mammal (e.g., a human) having cancer (e.g., lymphoma) can be identified as being likely to experience cancer relapse based, at least in part, on the presence of an elevated level of Fusobacteria, a reduced level of Akkermansia, and/or a reduced level of Barnesiellaceae in the mammal's gut microbiome. For example, a mammal (e.g., a human) having cancer (e.g., lymphoma) and having an elevated level of Fusobacteria, a reduced level of Akkermansia, and/or a reduced level of Barnesiellaceae in a sample (e.g., a stool sample) obtained from the mammal can be identified as being likely to experience cancer relapse. In some cases, a mammal (e.g., a human) having cancer (e.g., lymphoma) can be identified as being likely to not experience cancer relapse based, at least in part, on the absence of an elevated level of Fusobacteria, the absence of a reduced level of Akkermansia, and/or the absence of a reduced level of Barnesiellaceae in the mammal's gut microbiome. For example, a mammal (e.g., a human) having cancer (e.g., lymphoma) and lacking an elevated level of Fusobacteria, a reduced level of Akkermansia, and/or a reduced level of Barnesiellaceae in a sample (e.g., a stool sample) obtained from the mammal can be identified as being likely to not experience cancer relapse.

In some cases, the presence, absence, or level of one or more microbes (e.g., Fusobacteria, Akkermansia, and/or Barnesiellaceae) in a gut microbiome of a mammal (e.g., a human) having cancer (e.g., lymphoma) can be used to predict survival of the mammal. In some cases, a mammal (e.g., a human) having cancer (e.g., lymphoma) can be identified as being likely to experience shorter survival (e.g., overall survival (OS), event-free survival (EFS), and cancer-free survival) based, at least in part, on the presence of an elevated level of Fusobacteria, a reduced level of Akkermansia, and/or a reduced level of Barnesiellaceae in the mammal's gut microbiome. For example, a mammal (e.g., a human) having cancer (e.g., lymphoma) and having an elevated level of Fusobacteria, a reduced level of Akkermansia, and/or a reduced level of Barnesiellaceae in a sample (e.g., a stool sample) obtained from the mammal can be identified as being likely to survive for from about 2 months to about 24 months. In some cases, a mammal (e.g., a human) having cancer (e.g., lymphoma) can be identified as being likely to experience longer survival (e.g., OS, EFS, and cancer-free survival) based, at least in part, on the absence of an elevated level of Fusobacteria, the absence of a reduced level of Akkermansia, and/or the absence of a reduced level of Barnesiellaceae in the mammal's gut microbiome. For example, a mammal (e.g., a human) having cancer (e.g., lymphoma) and lacking an elevated level of Fusobacteria, a reduced level of Akkermansia, and/or a reduced level of Barnesiellaceae in a sample (e.g., a stool sample) obtained from the mammal can be identified as being likely to survive for from about 10 months to about 10 years.

In some cases, the presence, absence, or level of one or more microbes (e.g., Fusobacteria, Akkermansia, and/or Barnesiellaceae) in a gut microbiome of a mammal (e.g., a human) having cancer (e.g., lymphoma) can be used to select a cancer treatment. In some cases, a mammal (e.g., a human) having cancer (e.g., lymphoma) and identified as having elevated level of Fusobacteria, a reduced level of Akkermansia, and/or a reduced level of Barnesiellaceae in the mammal's gut microbiome can be selected for a cancer treatment based, at least in part, on the presence of an elevated level of Fusobacteria, a reduced level of Akkermansia, and/or a reduced level of Barnesiellaceae in the mammal's gut microbiome. For example, a mammal (e.g., a human) having cancer (e.g., lymphoma) and having an elevated level of Fusobacteria, a reduced level of Akkermansia, and/or a reduced level of Barnesiellaceae in a sample (e.g., a stool sample) obtained from the mammal can be selected for treatment with one or more agents that can reduce Fusobacteria levels within the gut microbiome (e.g., an antibiotic composition that targets Fusobacteria) and/or one or more agents that can increase Akkermansia and/or Barnesiellaceae levels within the gut microbiome (e.g., a probiotic or composition including live Akkermansia and/or live Barnesiellaceae) to the mammal to treat the mammal.

In some cases, a mammal (e.g., a human) having cancer (e.g., lymphoma) can be administered one or more cancer treatments selected as described herein (e.g., based, at least in part, on the presence of an elevated level of Fusobacteria, a reduced level of Akkermansia, and/or a reduced level of Barnesiellaceae in the mammal's gut microbiome). In some cases, a mammal (e.g., a human) having cancer (e.g., lymphoma) and identified as having elevated level of Fusobacteria, a reduced level of Akkermansia, and/or a reduced level of Barnesiellaceae in the mammal's gut microbiome can be administered or instructed to self-administer one or more (e.g., one, two, three, four, five, or more) agents and/or be subjected to one or more (e.g., one, two, three, or more) therapies that can reduce Fusobacteria levels within the gut microbiome. For example, a mammal (e.g., a human) having cancer (e.g., lymphoma) and having an elevated level of Fusobacteria, a reduced level of Akkermansia, and/or a reduced level of Barnesiellaceae in a sample (e.g., a stool sample) obtained from the mammal can be administered or instructed to self-administer one or more agents that can reduce Fusobacteria levels within the gut microbiome.

An agent that can reduce Fusobacteria levels within the gut microbiome can be any appropriate type of agent (e.g., small molecules, polypeptides, and nucleic acids such as DNA, RNA, and DNA/RNA hybrids). In some cases, an agent that can reduce Fusobacteria levels within the gut microbiome can be an antibiotic composition (e.g., an antibiotic composition that kills Fusobacteria). An antibiotic can be a narrow-spectrum antibiotic (e.g., an antibiotic composition that targets limited species of bacteria) or a broad-spectrum antibiotic (e.g., an antibiotic composition that targets broad classes of bacteria, such as gram-negative bacteria or gram-positive bacteria). Examples of agents that can reduce Fusobacteria levels within the gut microbiome include, without limitation, metronidazole, probiotics, and synbiotics. Examples of therapies that can reduce Fusobacteria levels within the gut microbiome include, without limitation, phage therapy, diet manipulation, and stool transplants.

In some cases, a mammal (e.g., a human) having cancer (e.g., lymphoma) and identified as having elevated level of Fusobacteria, a reduced level of Akkermansia, and/or a reduced level of Barnesiellaceae in the mammal's gut microbiome can be administered or instructed to self-administer one or more (e.g., one, two, three, four, five, or more) agents and/or be subjected to one or more (e.g., one, two, three, or more) therapies that can increase Akkermansia levels within the gut microbiome. For example, a mammal (e.g., a human) having cancer (e.g., lymphoma) and having an elevated level of Fusobacteria, a reduced level of Akkermansia, and/or a reduced level of Barnesiellaceae in a sample (e.g., a stool sample) obtained from the mammal can be administered or instructed to self-administer one or more agents that can increase Akkermansia levels within the gut microbiome.

An agent that can increase Akkermansia levels within a gut microbiome can be any appropriate type of agent. In some cases, an agent that can increase Akkermansia levels within a gut microbiome can be a composition (e.g., a probiotic composition) including viable Akkermansia (e.g., including one or more viable Akkermansia sp.). Examples of Akkermansia species that can be included in a composition that can be used as described herein to increase Akkermansia levels within the gut microbiome include, without limitation, A. muciniphila, A. muciniphilia, and A. glycaniphila. In some cases, an Akkermansia species that can be included in a composition that can be used as described herein to increase Akkermansia levels within the gut microbiome can be as deposited with the Agricultural Research Service (ARS) Patent Culture Collection under NRRL number B-68146. For example, a composition containing an Akkermansia species having phenotypic and/or genotypic characteristics substantially similar to those of the Akkermansia species deposited with the ARS Patent Culture Collection under NRRL number B-68146 can be administered to a mammal (e.g., a human) having cancer (e.g., lymphoma) to treat that cancer. In some cases, a composition containing viable cells of the Akkermansia deposited with the ARS Patent Culture Collection under NRRL number B-68146 can be administered to a mammal (e.g., a human) having cancer (e.g., lymphoma) to treat that cancer. In some cases, an Akkermansia species that can be included in a composition that can be used as described herein to increase Akkermansia levels within the gut microbiome can be as deposited with the ARS Patent Culture Collection under NRRL number B-68147. For example, a composition containing an Akkermansia species having phenotypic and/or genotypic characteristics substantially similar to those of the Akkermansia species deposited with the ARS Patent Culture Collection under NRRL number B-68147 can be administered to a mammal (e.g., a human) having cancer (e.g., lymphoma) to treat that cancer. In some cases, a composition containing viable cells of the Akkermansia deposited with the ARS Patent Culture Collection under NRRL number B-68147 can be administered to a mammal (e.g., a human) having cancer (e.g., lymphoma) to treat that cancer. A composition (e.g., a probiotic composition) including viable Akkermansia (e.g., including one or more viable Akkermansia sp.) can include any appropriate amount of viable Akkermansia. In some cases, a composition (e.g., a probiotic composition) including viable Akkermansia (e.g., including one or more viable Akkermansia sp.) can include at least 10⁹ colony forming units (CFUs) of viable Akkermansia. For example, a composition (e.g., a probiotic composition) including viable Akkermansia (e.g., including one or more viable Akkermansia sp.) can include from about 10⁹ CFUs to about 10¹¹ CFUs of viable Akkermansia.

In some cases, a mammal (e.g., a human) having cancer (e.g., lymphoma) and identified as having elevated level of Fusobacteria, a reduced level of Akkermansia, and/or a reduced level of Barnesiellaceae in the mammal's gut microbiome can be administered or instructed to self-administer one or more (e.g., one, two, three, four, five, or more) agents and/or be subjected to one or more (e.g., one, two, three, or more) therapies that can increase Barnesiellaceae levels within the gut microbiome. For example, a mammal (e.g., a human) having cancer (e.g., lymphoma) and having an elevated level of Fusobacteria, a reduced level of Akkermansia, and/or a reduced level of Barnesiellaceae in a sample (e.g., a stool sample) obtained from the mammal can be administered or instructed to self-administer one or more agents that can increase Barnesiellaceae levels within the gut microbiome.

An agent that can increase Barnesiellaceae levels within a gut microbiome can be any appropriate type of agent. In some cases, an agent that can increase Barnesiellaceae levels within a gut microbiome can be a composition (e.g., a probiotic composition) including viable Barnesiellaceae (e.g., including one or more viable Barnesiellaceae sp.). Examples of Barnesiellaceae species that can be included in a composition that can be used as described herein to increase Barnesiellaceae levels within the gut microbiome include, without limitation, B. viscericola, B. intestinihominis, B. sp904502265, B. excrementigallinarum, B. excrementipullorum, B. excrementavium, B. merdipullorum, and B. merdigallinarum. In some cases, a Barnesiellaceae species that can be included in a composition that can be used as described herein to increase Barnesiellaceae levels within the gut microbiome can be as deposited with the ARS Patent Culture Collection under NRRL number B-68145. For example, a composition containing an Barnesiellaceae species having phenotypic and/or genotypic characteristics substantially similar to those of the Barnesiellaceae species deposited with the ARS Patent Culture Collection under NRRL number B-68145 can be administered to a mammal (e.g., a human) having cancer (e.g., lymphoma) to treat that cancer. In some cases, a composition containing viable cells of the Barnesiellaceae deposited with the ARS Patent Culture Collection under NRRL number B-68145 can be administered to a mammal (e.g., a human) having cancer (e.g., lymphoma) to treat that cancer. A composition (e.g., a probiotic composition) including viable Barnesiellaceae (e.g., including one or more viable Barnesiellaceae sp.) can include any appropriate amount of viable Barnesiellaceae. In some cases, a composition (e.g., a probiotic composition) including viable Barnesiellaceae (e.g., including one or more viable Barnesiellaceae sp.) can include at least 10⁹ CFUs of viable Barnesiellaceae. For example, a composition (e.g., a probiotic composition) including viable Barnesiellaceae (e.g., including one or more viable Barnesiellaceae sp.) can include from about 10⁹ CFUs to about 10¹¹ CFUs of viable Barnesiellaceae.

A composition (e.g., a probiotic composition) that includes one or more Akkermansia sp. and/or one or more Barnesiellaceae sp. can include one or more additional viable microbes (e.g., viable bacteria). Examples of microbes that can be included in a composition that includes one or more Akkermansia sp. and/or one or more Barnesiellaceae sp. include, without limitation, Lactobacillis species, Bifidobacteria, Enterococcus faeceum (e.g., E. faecium SF66 and E. faecium SF68), and Saccharomyces cerevisiae (e.g., S. cerevisiae subsp. Boulardii).

In some cases, a mammal (e.g., a human) having cancer (e.g., lymphoma) and identified as having elevated level of Fusobacteria, a reduced level of Akkermansia, and/or a reduced level of Barnesiellaceae in the mammal's gut microbiome can be administered or instructed to self-administer one or more (e.g., one, two, three, four, five, or more) agents and/or be subjected to one or more (e.g., one, two, three, or more) therapies that can reduce Fusobacteria levels within the gut microbiome, can be administered or instructed to self-administer one or more (e.g., one, two, three, four, five, or more) agents and/or be subjected to one or more (e.g., one, two, three, or more) therapies that can increase Akkermansia levels within the gut microbiome, and/or can be administered or instructed to self-administer one or more (e.g., one, two, three, four, five, or more) agents and/or be subjected to one or more (e.g., one, two, three, or more) therapies that can increase Barnesiellaceae levels within the gut microbiome. For example, a mammal (e.g., a human) having cancer (e.g., lymphoma) and having an elevated level of Fusobacteria, a reduced level of Akkermansia, and a reduced level of Barnesiellaceae in a sample (e.g., a stool sample) obtained from the mammal can be administered or instructed to self-administer one or more agents that can reduce Fusobacteria levels within the gut microbiome without reducing Akkermansia levels within the gut microbiome and/or without reducing Barnesiellaceae levels within the gut microbiome. For example, a mammal (e.g., a human) having cancer (e.g., lymphoma) and having an elevated level of Fusobacteria, a reduced level of Akkermansia, and/or a reduced level of Barnesiellaceae in a sample (e.g., a stool sample) obtained from the mammal can be administered or instructed to self-administer one or more agents that can increase Akkermansia levels within the gut microbiome without increasing Fusobacteria levels within the gut microbiome and/or without reducing Barnesielaceae levels within the gut microbiome. For example, a mammal (e.g., a human) having cancer (e.g., lymphoma) and having an elevated level of Fusobacteria, a reduced level of Akkermansia, and/or a reduced level of Barnesiellaceae in a sample (e.g., a stool sample) obtained from the mammal can be administered or instructed to self-administer one or more agents that can increase Barnesiellaceae levels within the gut microbiome without increasing Fusobacteria levels within the gut microbiome and/or without reducing Akkermansia levels within the gut microbiome.

In some cases, a mammal (e.g., a human) having cancer (e.g., lymphoma) and identified as having elevated level of Fusobacteria, a reduced level of Akkermansia, and/or a reduced level of Barnesiellaceae in the mammal's gut microbiome can be administered or instructed to self-administer one or more (e.g., one, two, three, four, five, or more) agents and/or be subjected to one or more (e.g., one, two, three, or more) therapies that can reduce Fusobacteria levels within the gut microbiome, and can subsequently be administered or instructed to self-administer one or more (e.g., one, two, three, four, five, or more) agents and/or be subjected to one or more (e.g., one, two, three, or more) therapies that can increase Akkermansia levels within the gut microbiome, and/or can be administered or instructed to self-administer one or more (e.g., one, two, three, four, five, or more) agents and/or be subjected to one or more (e.g., one, two, three, or more) therapies that can increase Barnesiellaceae levels within the gut microbiome.

In some cases, when treating a mammal (e.g., a human) having cancer (e.g., lymphoma) as described herein, the treatment can be effective to improve survival of the mammal. For example, the methods and materials described herein can be used to improve EFS (e.g., cancer-free survival). For example, the methods and materials described herein can be used to improve OS. In some cases, the methods and materials described herein can be used to improve the survival of a mammal having cancer (e.g., lymphoma) by, for example, 10, 20, 30, 40, 50, 60, 70, 80, 90, 95, or more percent. For example, the methods and materials described herein can be used to improve the survival of a mammal having cancer (e.g., lymphoma) by, for example, at least 6 months (e.g., about 6 months, about 8 months, about 10 months, about 1 year, about 1.5 years, about 2 years, about 2.5 years, or about 3 years).

In some cases, when treating a mammal (e.g., a human) having cancer (e.g., lymphoma) as described herein, the treatment can be effective to treat the cancer. For example, the number of cancer cells present within a mammal can be reduced using the methods and materials described herein.

In some cases, the number of cancer cells present within a mammal can be reduced using the methods and materials described herein. For example, the methods and materials described herein can be used to reduce the number of cancer cells present within a mammal having cancer by, for example, 10, 20, 30, 40, 50, 60, 70, 80, 90, 95, or more percent. In some cases, the size (e.g., volume) of one or more tumors present within a mammal can be reduced using the methods and materials described herein. For example, the methods and materials described herein can be used to reduce the size of one or more tumors present within a mammal having cancer by, for example, 10, 20, 30, 40, 50, 60, 70, 80, 90, 95, or more percent. In some cases, the size (e.g., volume) of one or more tumors present within a mammal does not increase.

In some cases, when treating a mammal (e.g., a human) having cancer (e.g., lymphoma) as described herein, the treatment can be effective to reduce one or more symptoms of the cancer. Examples of symptoms of lymphoma include, without limitation, painless swelling of lymph nodes (e.g., lymph nodes in the neck, axilla, or groin), persistent fatigue, fever, night sweats, shortness of breath, unexplained weight loss, and itchy skin. For example, the methods and materials described herein can be used to reduce one or more symptoms within a mammal having cancer (e.g., lymphoma) by, for example, 10, 20, 30, 40, 50, 60, 70, 80, 90, 95, or more percent.

In some cases, when treating a mammal (e.g., a human) having cancer (e.g., lymphoma) and having one or more GI symptoms as described herein, the treatment can be effective to reduce one or more GI symptoms. Examples of GI symptoms include, without limitation, without limitation, abnormal GI tract motility, abnormal GI tract sensation, and brain-gut GI tract dysfunction. For example, the methods and materials described herein can be used to reduce one or more GI symptoms within a mammal having cancer (e.g., lymphoma) and having one or more GI symptoms by, for example, 10, 20, 30, 40, 50, 60, 70, 80, 90, 95, or more percent.

In some cases, when treating a mammal (e.g., a human) having cancer (e.g., lymphoma) and having one or more GI symptoms as described herein, the treatment can be effective to reduce one or more side-effects of a cancer treatment being administered to the mammal. Examples of side-effects associated with a cancer treatment being administered to a mammal (e.g., a human) having cancer (e.g., lymphoma) include, without limitation, without limitation, diarrhea, constipation, infection, nausea, vomiting, loss of appetite, and weight loss. For example, the methods and materials described herein can be used to reduce one or more side-effects associated with a cancer treatment being administered to a mammal (e.g., a human) having cancer (e.g., lymphoma) within the mammal by, for example, 10, 20, 30, 40, 50, 60, 70, 80, 90, 95, or more percent.

In some cases, when treating a mammal (e.g., a human) having cancer (e.g., lymphoma) as described herein (e.g., by administering one or more agents that can reduce Fusobacteria levels within the gut microbiome and/or one or more agents that can increase Akkermansia and/or Barnesiellaceae levels within the gut microbiome to the mammal), the one or more agents that can reduce Fusobacteria levels within the gut microbiome and/or one or more agents that can increase Akkermansia and/or Barnesiellaceae levels within the gut microbiome to the mammal can be the sole active agent(s) administered to the mammal to treat the cancer. For example, one or more agents that can reduce Fusobacteria levels within the gut microbiome and/or one or more agents that can increase Akkermansia and/or Barnesiellaceae levels within the gut microbiome of a mammal can be administered to a mammal (e.g., a human) having a cancer (e.g., a lymphoma) that does not require immediate treatment such as a cancer that is in remission (e.g., as a maintenance therapy to delay or prevent the return of the cancer).

In some cases, when treating a mammal (e.g., a human) having cancer (e.g., lymphoma) as described herein (e.g., by administering one or more agents that can reduce Fusobacteria levels within the gut microbiome and/or one or more agents that can increase Akkermansia and/or Barnesiellaceae levels within the gut microbiome to the mammal), the mammal also can be treated with one or more additional agents or therapies used to treat cancer. In some cases, an additional agent used to treat cancer can be an alkylating agent. In some cases, an additional agent used to treat cancer can be a corticosteroid. In some cases, an additional agent used to treat cancer can be a platinum drug. In some cases, an additional agent used to treat cancer can be a purine analog. In some cases, an additional agent used to treat cancer can be an anti-metabolite. In some cases, an additional agent used to treat cancer can be an anthracycline. In some cases, an additional agent used to treat cancer can be chemotherapeutic agent. In some cases, an additional agent used to treat cancer can be an immunotherapy (e.g., can include one or more monoclonal antibodies). In some cases, an additional agent used to treat cancer can be an immune checkpoint inhibitor. In some cases, an additional agent used to treat cancer can be a signal transduction inhibitor. In some cases, an additional agent used to treat cancer can be a BTK inhibitor. Examples of agents (e.g., anti-cancer agents) that can be administered to mammal (e.g., a human) having cancer (e.g., lymphoma) together with one or more agents that can reduce Fusobacteria levels within the gut microbiome and/or one or more agents that can increase Akkermansia and/or Barnesiellaceae levels within the gut microbiome include, without limitation, cyclophosphamide, chlorambucil, bendamustine, ifosfamide, prednisone, dexamethasone, cisplatin, carboplatin, oxaliplatin, fludarabine, pentostatin, cladribine (2-CdA), cytarabine (ara-C), gemcitabine, methotrexate, pralatrexate, doxorubicin (adriamycin), liposomal doxorubicin (caelyx), vincristine, mitoxantrone, etoposide (VP-16), bleomycin, and any combinations thereof. In cases where one or more agents that can reduce Fusobacteria levels within the gut microbiome and/or one or more agents that can increase Akkermansia and/or Barnesiellaceae levels within the gut microbiome are used in combination with additional agents used to treat cancer, the one or more agents that can reduce Fusobacteria levels within the gut microbiome and/or one or more agents that can increase Akkermansia and/or Barnesiellaceae levels within the gut microbiome can be administered at the same time (e.g., in a single composition containing both one or more agents that can reduce Fusobacteria levels within the gut microbiome and/or one or more agents that can increase Akkermansia and/or Barnesiellaceae levels within the gut microbiome and the one or more additional agents) or independently. For example, one or more agents that can reduce Fusobacteria levels within the gut microbiome and/or one or more agents that can increase Akkermansia and/or Barnesiellaceae levels within the gut microbiome can be administered first, and the one or more additional agents administered second, or vice versa. Examples of therapies that can be used to treat cancer include, without limitation, surgery, radiation therapies, immunotherapies (e.g., immunotherapies including monoclonal antibodies, antibody-drug conjugates, radioactive antibodies, immune checkpoint inhibitors, and/or bispecific antibodies), bone marrow transplants, and cell therapies such as a chimeric antigen receptor (CAR)-T cell therapy. In cases where one or more agents that can reduce Fusobacteria levels within the gut microbiome and/or one or more agents that can increase Akkermansia and/or Barnesiellaceae levels within the gut microbiome are used in combination with one or more additional therapies used to treat cancer, the one or more additional therapies can be performed at the same time or independently of the administration of one or more agents that can reduce Fusobacteria levels within the gut microbiome and/or one or more agents that can increase Akkermansia and/or Barnesiellaceae levels within the gut microbiome. For example, the one or more agents that can reduce Fusobacteria levels within the gut microbiome and/or one or more agents that can increase Akkermansia and/or Barnesiellaceae levels within the gut microbiome can be administered before, during, or after the one or more additional therapies are performed.

When one or more agents that can reduce Fusobacteria levels within the gut microbiome and/or one or more agents that can increase Akkermansia and/or Barnesiellaceae levels within the gut microbiome are administered prior to administering one or more additional agents or therapies used to treat cancer, the one or more agents that can reduce Fusobacteria levels within the gut microbiome and/or one or more agents that can increase Akkermansia and/or Barnesiellaceae levels within the gut microbiome can be effective to enhance the outcome of treatment with the one or more additional agents or therapies used to treat cancer. For example, one or more agents that can reduce Fusobacteria levels within the gut microbiome and/or one or more agents that can increase Akkermansia and/or Barnesiellaceae levels within the gut microbiome can be administered to a mammal (e.g., a human) having cancer (e.g., lymphoma) prior to subjecting the mammal to one or more cell therapies (e.g., CAR-T cell therapies) to enhance the outcome of the one or more cell therapies. For example, one or more agents that can reduce Fusobacteria levels within the gut microbiome and/or one or more agents that can increase Akkermansia and/or Barnesiellaceae levels within the gut microbiome can be administered to a mammal (e.g., a human) having cancer (e.g., lymphoma) prior to subjecting the mammal to a bone marrow transplant to enhance the outcome of the bone marrow transplant.

In certain instances, a course of treatment and the severity of one or more symptoms (e.g., cancer symptoms such as lymphoma symptoms and/or GI symptoms experienced by a mammal having cancer) can be monitored. For example, the severity of one or more symptoms (e.g., cancer symptoms such as lymphoma symptoms and/or GI symptoms experienced by a mammal having cancer) can be monitored over the course of treatment to determine whether or not the treatment is effective and/or remains effective over time. Any appropriate method can be used to determine whether or not the severity of a symptom is reduced. In some cases, the severity of one or more symptoms (e.g., cancer symptoms such as lymphoma symptoms and/or GI symptoms experienced by a mammal having cancer) can be assessed at different time points. For example, the severity of one or more symptoms (e.g., cancer symptoms such as lymphoma symptoms and/or GI symptoms experienced by a mammal having cancer) can be monitored from about 7 days to about 24 weeks after the mammal has been administered (or has self-administered) one or more agents that increase Akkermansia and/or Barnesiellaceae levels within the gut microbiome to determine whether the levels of Akkermansia and/or Barnesiellaceae within the gut microbiome of the mammal have increased. In cases where the levels of Akkermansia and/or Barnesiellaceae within the gut microbiome of the mammal have increased, the treatment course can enter a rest period or can be ended. In cases where the levels of Akkermansia and/or Barnesiellaceae within the gut microbiome of the mammal have not increased, the treatment course can be continued and the mammal can be administered (or has self-administered) one or more agents that increase Akkermansia and/or Barnesiellaceae levels within the gut microbiome. When the course of treatment is continued, the one or more agents that increase Akkermansia and/or Barnesiellaceae levels within the gut microbiome can be same as the first administration or can be different from the first administration.

In some cases, the severity of one or more symptoms (e.g., cancer symptoms such as lymphoma symptoms and/or GI symptoms experienced by a mammal having cancer) can be monitored from about 7 days to about 24 weeks after the mammal has been administered (or has self-administered) one or more agents that reduce Fusobacteria levels within the gut microbiome to determine whether the levels of Fusobacteria within the gut microbiome of the mammal have been reduced. In cases where the levels of Fusobacteria within the gut microbiome of the mammal have been reduced, the treatment course can enter a rest period or can be ended. In cases where the levels of Fusobacteria within the gut microbiome of the mammal have not been reduced, the treatment course can be continued and the mammal can be administered (or has self-administered) one or more agents that reduce Fusobacteria levels within the gut microbiome. When the course of treatment is continued, the one or more agents that reduce Fusobacteria levels within the gut microbiome can be same as the first administration or can be different from the first administration.

In some cases, treatment of a mammal with one or more agents that can reduce Fusobacteria levels within the gut microbiome and/or one or more agents that can increase Akkermansia and/or Barnesiellaceae levels within the gut microbiome can occur even after treatment of the mammal for cancer has stopped.

In some cases, an agent or therapy that can reduce Fusobacteria levels within the gut microbiome (e.g., a small molecule, a polypeptide, a nucleic acid such as a DNA, RNA, or DNA/RNA hybrid, an antibiotic composition such as an antibiotic composition that kills Fusobacteria, an antibiotic composition containing a narrow-spectrum antibiotic, or an antibiotic composition containing a broad-spectrum antibiotic, an agent such as metronidazole, a probiotic, or a symbiotic, or a therapy such as phage therapy, diet manipulation, or a stool transplant) can be administered to a mammal after treatment of the mammal for cancer has stopped. For example, an agent or therapy that can reduce Fusobacteria levels within the gut microbiome can be administered to a mammal having cancer with one or more additional agents or therapies used to treat the cancer, and treatment with the agent or therapy that can reduce Fusobacteria levels within the gut microbiome can continue (e.g., as supportive care) after treatment with the one or more additional agents or therapies has stopped.

In some cases, an agent that can increase Akkermansia levels within a gut microbiome (e.g., a composition containing viable Akkermansia, such as one or more viable Akkermansia sp. selected from A. muciniphila, A. muciniphilia, and A. glycaniphila, an Akkermansia species deposited with the Agricultural Research Service (ARS) Patent Culture Collection under NRRL number B-68146, an Akkermansia species having phenotypic and/or genotypic characteristics substantially similar to those of the Akkermansia species deposited with the ARS Patent Culture Collection under NRRL number B-68146, an Akkermansia species deposited with the ARS Patent Culture Collection under NRRL number B-68147, or an Akkermansia species having phenotypic and/or genotypic characteristics substantially similar to those of the Akkermansia species deposited with the ARS Patent Culture Collection under NRRL number B-68147) can be administered to a mammal after treatment of the mammal for cancer has stopped. For example, an agent or therapy that can increase Akkermansia levels within the gut microbiome can be administered to a mammal having cancer with one or more additional agents or therapies used to treat the cancer, and treatment with the agent or therapy that can increase Akkermansia levels within the gut microbiome can continue (e.g., as supportive care) after treatment with the one or more additional agents or therapies has stopped.

In some cases, an agent that can increase Barnesiellaceae levels within a gut microbiome (e.g., a composition containing viable Barnesiellaceae, such as one or more viable Barnesiellaceae sp. selected from B. viscericola, B. intestinihominis, B. sp904502265, B. excrementigallinarum, B. excrementipullorum, B. excrementavium, B. merdipullorum, and B. merdigallinarum, a Barnesiellaceae species deposited with the ARS Patent Culture Collection under NRRL number B-68145, or a composition containing an Barnesiellaceae species having phenotypic and/or genotypic characteristics substantially similar to those of the Barnesiellaceae species deposited with the ARS Patent Culture Collection under NRRL number B-68145) can be administered to a mammal after treatment of the mammal for cancer has stopped. For example, an agent or therapy that can increase Barnesiellaceae levels within the gut microbiome can be administered to a mammal having cancer with one or more additional agents or therapies used to treat the cancer, and treatment with the agent or therapy that can increase Barnesiellaceae levels within the gut microbiome can continue (e.g., as supportive care) after treatment with the one or more additional agents or therapies has stopped.

The invention will be further described in the following examples, which do not limit the scope of the invention described in the claims.

### EXAMPLES

### Example 1: Gut Microbiome Content as a Predictor of Outcome in Cancer

This Example describes the identification of a gut microbiome that can be used to predict event-free survival and overall survival in lymphoma patients.

### Methods

Stool samples were provided and cryopreserved from patients with new, untreated lymphoma. A control stool sample was also requested from a person living in the same household (household control). Patients were followed for response to treatment and survival as well as toxicity (FIG. 1A). A schematic for the data analysis is shown in FIG. 1B.

### Patient Characteristics

Stool samples from 458 cases of new, untreated lymphoma were analyzed. Patients provided a stool sample prior to treatment start and a blood sample. All patients had routine standard of care tumor imaging, treatment and were followed for an event (treatment failure and death due to any cause). Of the 458 lymphoma cases, 51 had known gastrointestinal involvement with lymphoma. In 140 cases, a household control was obtained and analyzed. Thus, a total of 598 samples were analyzed. Characteristics of lymphoma patients and of matched controls are shown in Table 1 and Table 2.

TABLE 1
Patient Characteristics of the Lymphoma Microbiome study.

| | Control | | Case | | Total | | |
|---|---|---|---|---|---|---|---|
| | (N = 140) | | (N = 458) | | (N = 598) | | p value |
| Gender | | | | | | | <0.001 |
| N-Missing | 30 | | 0 | | 30 | | |
| Female | 73 | (66.4%) | 181 | (39.5%) | 254 | (44.7%) | |
| Male | 37 | (33.6%) | 277 | (60.5%) | 314 | (55.3%) | |
| Age | | | | | | | 0.840 |
| N-Missing | 30 | | 1 | | 31 | | |
| Mean (SD) | 61.79 | (13.08) | 61.51 | (12.90) | 61.57 | (12.92) | |

| Range | 27.00-90.00 | 20.00-95.00 | | 20.00-95.00 | |
|---|---|---|---|---|---|
| BMI | | | | | |
| N-Missing | 140 | 16 | | 156 | |
| Mean (SD) | NA | 29.17 | (8.69) | 29.17 | (8.69) |

| | | | |
|---|---|---|---|
| Range | NA | 3.89-160.88 | 3.89-160.88 |

Abbreviations: BMI, body mass index.

TABLE 2
Lymphoma Characteristics.

Case (N = 458)

| | | |
|---|---|---|
| Lymphoma involvement of | | |
| the gastrointestinal tract | | |
| No | 407 | (88.9%) |
| Yes | 51 | (11.1%) |
| Types of Lymphoma | | |
| CLL/SLL | 7 | (1.5%) |
| Composites | 17 | (3.7%) |
| DLBCL | 113 | (24.7%) |
| FL | 118 | (25.8%) |
| HL | 35 | (7.6%) |
| MCL | 41 | (9.0%) |
| MZL | 58 | (12.7%) |
| Other NHL | 27 | (5.9%) |
| TCL | 35 | (7.6%) |
| WM | 7 | (1.5%) |
| General groupings of Lymphoma | | |
| Aggressive (DLBCL; Hodgkin; MCL; TCL) | 232 | (50.7%) |
| Low grade (CLL/SLL; MZL; WM) | 203 | (44.3%) |
| Other | 23 | (5.0%) |

Abbreviations: CLL/SLL, chronic lymphocytic leukemia/small lymphocytic lymphoma; DLBCL, diffuse large B-cell lymphoma; FL, follicular lymphoma; HL, Hodgkin lymphoma; MCL. Mantle cell lymphoma; MZL, marginal zone lymphoma; TCL, T-cell lymphoma; WM, Waldenstrom's macroglobulinemia.

### DNA Extraction Protocol for Stool Specimens

DNA was extracted from samples using a DNeasy® PowerSoil® HTP 96 Kit (Qiagen).

16S rRNVA Gene V3-V5 Targeted Sequencing Protocol to Determine Types of Microbes

### 1) Primer Sequences (16S-Specific Portion in Bold)

Meta_V3_F Nextera:
(SEQ ID NO: 1)
TCGTCGGCAGCGTCAGATGTGTATA
AGAGACAGCCTACGGGAGGCAGCAG
V5R_Nextera:
(SEQ ID NO: 2)
GTCTCGTGGGCTCGGAGATGTGTAT
AAGAGACAGCCGTCAATTCMTTTRA
GT

The indexing primers are as follows. This step adds both the index and the flowcell adapters. [i5] and [i7] refer to the index sequence codes used by Illumina. The p5 and p7 flow cell adapters are in bold.

Forward indexing primer:
(SEQ ID NO: 3)
AATGATACGGCGACCACCGAGATCTA
CAC[i5]TCGTCGGCAGCGTC
Reverse indexing primer:
(SEQ ID NO: 4)
CAAGCAGAAGACGGCATACGAGAT
[i7]GTCTCGTGGGCTCGG

### 2) Cycling Conditions

PCR reactions were performed using KAPA HiFidelity Hot Start Polymerase.
qPCR (using the Meta_V4_515F/Meta_V4_806R primer pair)
95° C.—5 minutes
35 cycles of
98° C.—20 seconds
55° C.—15 seconds
72° C.—1 minute
72° C.—5 minutes
Hold at 4° C.

After qPCR, samples were normalized to 167,000 molecules/μL. This was based on the volume of sample used for PCR1 (3 μL), so 500,000 molecules is roughly 10× the target sequencing coverage.
PCR 1 (using the Meta_V3_Nextera/V5R_Nextera primer pair)
95° C.—5 minutes
25 cycles of:
98° C.—20 seconds
55° C.—15 seconds
72° C.—1 minute
72° C.—5 minutes
Hold at 4° C.

After the first round of amplification, PCR 1 products were diluted 1:100 and 5 μL of 1:100 PCR 1 is used in the second PCR reaction.
PCR 2 (using different combinations of forward and reverse indexing primers)
95° C.—5 minutes
10 cycles of:
98° C.—20 seconds
55° C.—15 seconds
72° C.—1 minute
72° C.—5 minutes
Hold at 4° C.

### 3) Sequencing

Pooled sample was denatured with NaOH, diluted to 8 pM in Illumina's HT1 buffer, spiked with 10% PhiX, and heat denatured at 96 C for 2 minutes immediately prior to loading. A MiSeq 600 cycle v3 kit was used to sequence the sample. 4) Nextera adapter sequences (for post-run trimming):

Read 1:
(SEQ ID NO: 5)
CTGTCTCTTATACACATCTCCGAGCCCACG
AGACNNNNNNNNATCTCGTATGCCGTCTTC
TGCTTG
Read 2:
(SEQ ID NO: 6)
CTGTCTCTTATACACATCTGACGCTGCCGA
CGANNNNNNNNGTGTAGATCTCGGTGGTCG
CCGTATCATT

### Data Analysis—Bioinformatics and Biostatistics (FIG. 1B)

Paired R1 and R2 sequence reads were processed using DADA2. Taxonomy was assigned using the RDP classifier and the Silva database (v128). Genus-level abundances were calculated based on the assigned taxonomy. Geometric Mean of Pairwise Ratio (GMPR) normalization was used to normalize the abundance data. Cox proportional hazards (CPH) model was used to test the association between the square-root transformed genus abundance and the event-free survival (“coxph” function in the R “survival” package v3.1.11). Sex, age, BMI and aggressiveness and gut involvement were adjusted in the model. Taxa with prevalence less than 10% or with a maximum proportion less 0.2% were excluded from testing to reduce the number of the tests. False discovery rate control (BH procedure, “p.adjust” function in R “stats” package v3.6.3.) was used to correct for multiple testing. Two genera, Akkermansi and Barnesiella, were identified as at 10% false discovery rate. They were positively associated with event-free survival.

### Results

### Control Microbiota.

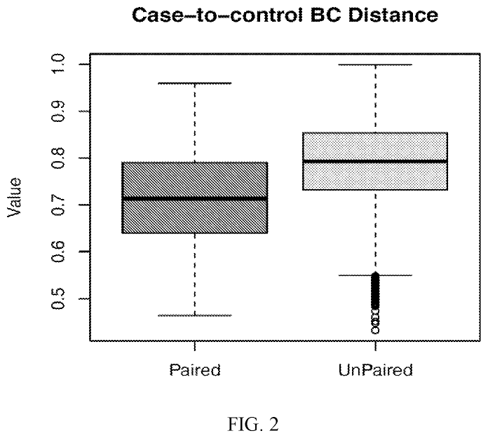

Control for confounding factors that influence gut microbiota composition like diet, household exposures such as pets, and environmental exposures can increase confidence in the obtained results. The microbiota of the household control was more similar to the matched patient than to the unmanaged cases (FIG. 2). This result validates the importance of having household controls.

### Gastrointestinal Microbiome Diversity

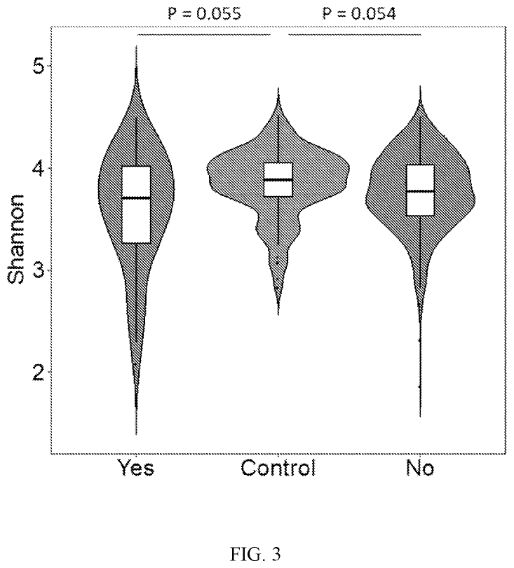

11% of patients had known involvement of the gastrointestinal tract with their cancer. The GMB of those patient's stools was less diverse than those lymphoma patients without involvement of the gastrointestinal tract (p=0.001, LMM) (FIG. 3). Lymphoma patients have lower microbial alpha diversity (Shannon index) irrespective of gut involvement (FIG. 3).

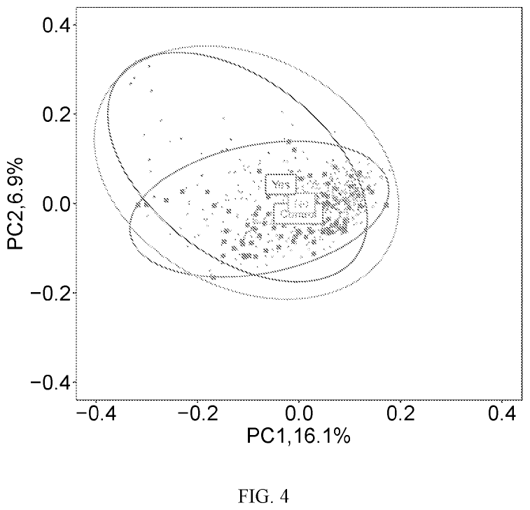

Lymphoma patients have significantly different microbial community structure compared to controls as indicated by the principal coordinate plot based on weighted UniFrac distance (FIG. 4, P=0.002, PERMANOVA).

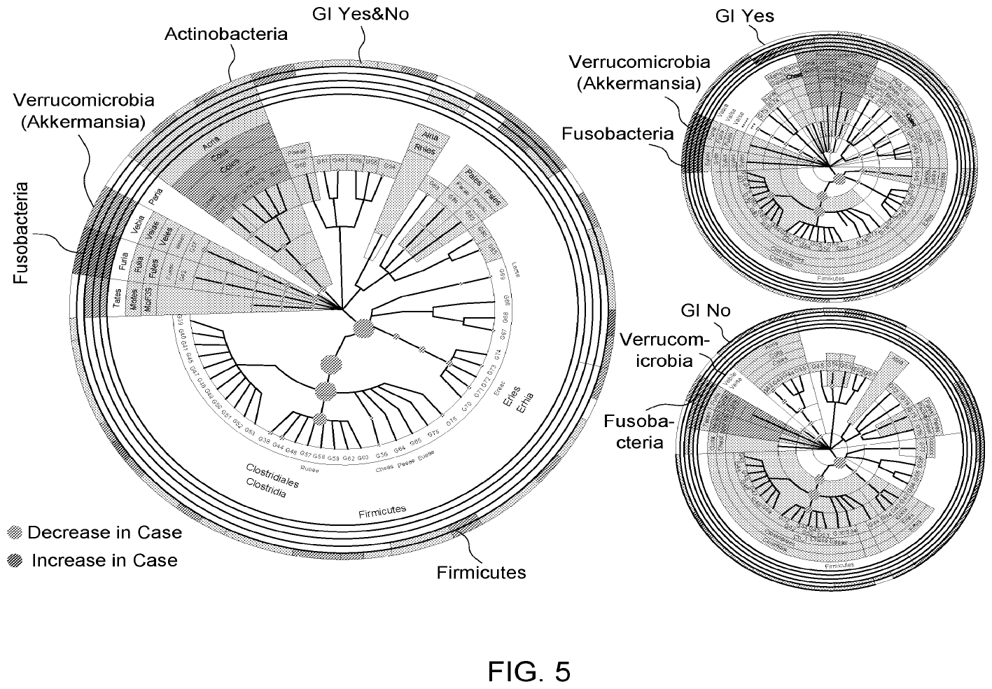

A few taxa at different taxonomic ranks were differentially abundant between lymphoma patients and controls (FIG. 5, FDR <=0.05). Particular genera of bacteria identified in FIG. 5 were further analyzed.

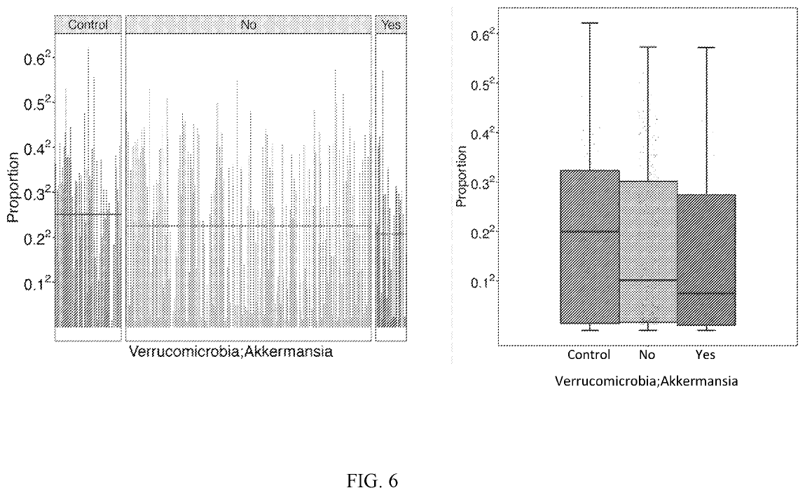

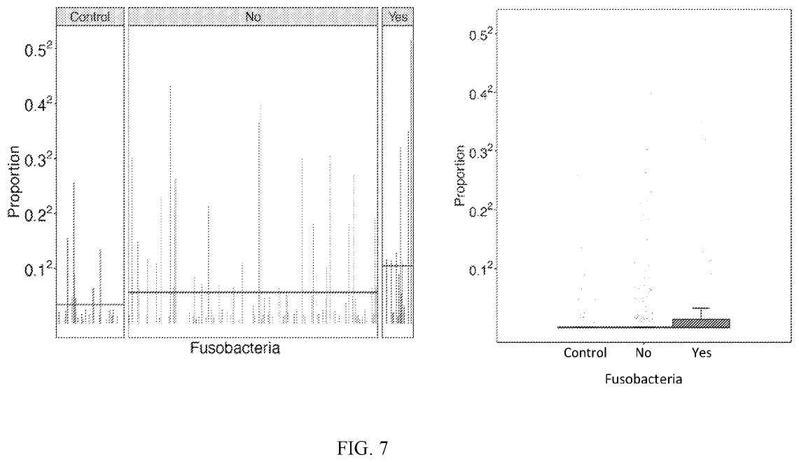

Particularly, Akkermansia was decreased in cases (FIG. 6), and Fusobacteria was increased in cases (FIG. 7).

These results demonstrate that lymphoma patients harbor a distinct microbiome.

### Survival

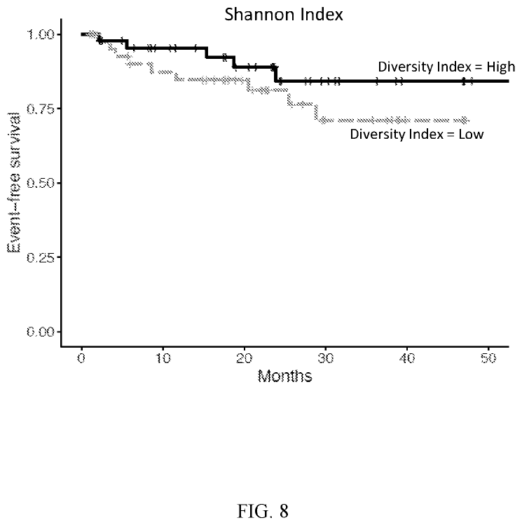

EFS of DLBCL lymphoma patients was evaluated. EFS was compared between patients with high Shannon index (>median) and low Shannon index (<=median) using Kaplan-Meir curves (P=0.046). EFS was shorter in those with DLBCL and a lower alpha diversity score (FIG. 8). These results demonstrate that EFS was associated with gut microbial diversity in DLBCL patients. MiRKAT-S testing the association between the survival times and the gut microbiome using Bray-Curtis distance adjusting disease subtype, sex, age, BMI, and Gut involvement demonstrated that both the overall survival (OS) and EFS were associated with microbiome composition and structure (P<0.05). The association was stronger in DLBCL patients.

Data for DLBCL and FL are shown in Table 3. Shannon index data were adjusted for disease subtype, sex, age, BMI, and GI involvement.

TABLE 3

| | | Observed | Shannon |
|---|---|---|---|
| All (n = 458) | EFS | 0.224 | 0.597 |
| | OS | 0.191 | 0.587 |
| DLBCL (n = 113) | EFS | 0.382 | 0.046 |
| | OS | 0.882 | 0.439 |
| FL (n = 118) | EFS | 0.893 | 0.346 |
| | OS | 0.300 | 0.493 |

MiRKAT-S was used for testing the association using Bray-Curtis distance adjusting disease subtype, sex, age, BMI, and Gut involvement. Data are shown in Table 4.

TABLE 4

| | All (n = 458) | DLBCL (n = 113) | FL (n = 118) |
|---|---|---|---|
| EFS | 0.035 | 0.002 | 0.570 |
| OS | 0.017 | 0.017 | 0.153 |

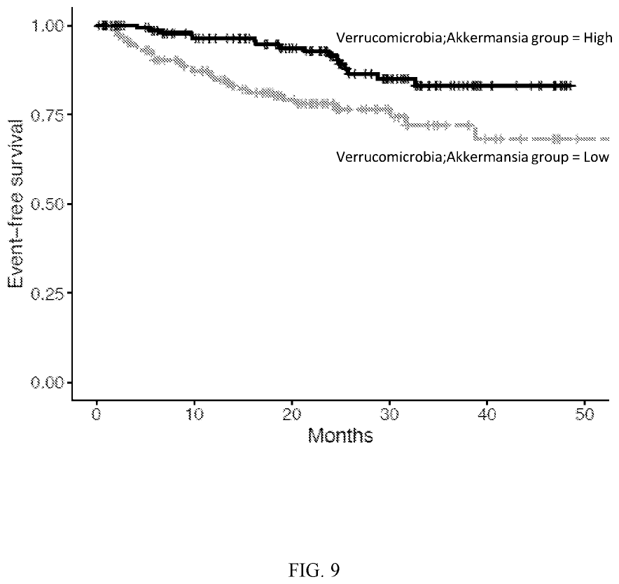

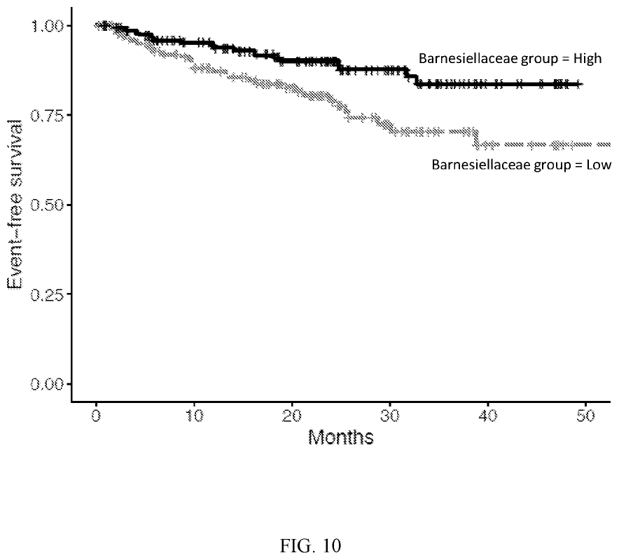

Survival was also evaluated based on the gastrointestinal microbiome diversity of the DLBCL patients. Higher amounts of Akkermansia (FIG. 9A), specifically, the species A. muciniphila (FIG. 9B) were associated with favorable survival. Elevated level of A. muciniphila is associated with improved outcomes in lymphoma patients. Higher amounts of Barnesiellaceae (FIG. 10) were associated with favorable survival.

These results demonstrate that elevated level of Akkermansia (e.g., A. muciniphila) and Barnesiellaceae are associated with improved outcomes in DLBCL patients.

### Example 2: Assessing Humans Having Lymphoma

A stool sample is obtained from a human having lymphoma. The obtained sample is used to identify the gut microbiome of the human having lymphoma. In some cases, a 16S RNA sequencing assay is performed to identify the gut microbiome of the human having lymphoma.

If a reduced level of Akkermansia (e.g., A. muciniphila) and/or Barnesiellaceae is identified in the gut microbiome of the human having lymphoma (e.g., as compared to controls such as household controls), the human having lymphoma is identified as being likely to have an inferior outcome (e.g., a poor treatment response, toxicity to a treatment, and/or a poor quality of life) or as being likely to have a shorter cancer free survival as compared to a human having lymphoma and not having a reduced level of Akkermansia and/or Barnesiellaceae.

If a reduced level of Akkermansia (e.g., A. muciniphila) and/or Barnesiellaceae is not identified in the gut microbiome of a human having lymphoma (e.g., as compared to controls such as household controls), the human having lymphoma is identified as being likely to have an improved outcome (e.g., as being unlikely to have a poor outcome or as being unlikely to have a shorter cancer free survival) as compared to a human having lymphoma and having a reduced level of Akkermansia and/or Barnesiellaceae.

### Example 3: Assessing Humans Having Lymphoma

A stool sample is obtained from a human having lymphoma. The obtained sample is used to identify the gut microbiome of the human having lymphoma. In some cases, a 16S RNA sequencing assay is performed to identify the gut microbiome of the human having lymphoma.

If an elevated level of Fusobacteria is identified in the gut microbiome of the human having lymphoma (e.g., as compared to controls such as household controls), the human having lymphoma is identified as being likely to have a poor outcome (e.g., a poor treatment response, toxicity to a treatment, and/or a poor quality of life) or as being likely to have a shorter cancer free survival as compared to a human having lymphoma and not having an elevated level of Fusobacteria.

If an elevated level of Fusobacteria is not identified in the gut microbiome of a human having lymphoma (e.g., as compared to controls such as household controls), the human having lymphoma is identified as being likely to have an improved outcome (e.g., as being unlikely to have a poor outcome or as being unlikely to have a shorter cancer free survival) as compared to a human having lymphoma and having an elevated level of Fusobacteria.

### Example 4—Treating Humans Having Lymphoma

A human having lymphoma and identified as having a reduced level of Akkermansia (e.g., A. muciniphila) and/or Barnesiellaceae in the gut microbiome is administered one or more agents that increase Akkermansia and/or Barnesielaceae levels within the gut microbiome (e.g., a composition, such as a prebiotic, synbiotic, or probiotic composition, including Akkermansia and/or Barnesiellaceae) as the sole active agent to treat the lymphoma (e.g., as a maintenance therapy to treat a lymphoma that does not require immediate treatment such as a lymphoma that is in remission). The treatment is used to improve survival (e.g., cancer free survival) of the human having lymphoma. In some cases, the treatment is used to prevent the need for administering a systemic lymphoma therapy to the human.

### Example 5—Treating Humans Having Lymphoma

A human having lymphoma and identified as having a reduced level of Akkermansia (e.g., A. muciniphila) and/or Barnesiellaceae in the gut microbiome is treated by administering one or more agents that increase Akkermansia and/or Barnesiellaceae levels within the gut microbiome (e.g., a probiotic or composition including Akkermansia and/or Barnesiellaceae) to the human in combination one or more lymphoma treatments (e.g., chemotherapies, immunotherapies, radiation therapies, bone marrow transplants, and/or cell therapies such as a CAR-T cell therapies). The treatment improves survival (e.g., cancer free survival) of the human having lymphoma.

### Example 6—Treating Humans Having Lymphoma

A human having lymphoma and identified as having an elevated level of Fusobacteria in the gut microbiome is administered one or more agents that reduce Fusobacteria levels within the gut microbiome (e.g., an antibiotic composition that targets Fusobacteria) as the sole active agent to treat the lymphoma (e.g., as a maintenance therapy to treat a lymphoma that does not require immediate treatment such as a lymphoma that is in remission). The treatment is used to improve survival (e.g., cancer free survival) of the human having lymphoma. In some cases, the treatment is used to prevent the need for administering a systemic lymphoma therapy to the human.

### Example 7—Treating Humans Having Lymphoma

A human having lymphoma and identified as having an elevated level of Fusobacteria in the gut microbiome is treated by administering one or more agents that reduce Fusobacteria levels within the gut microbiome (e.g., an antibiotic composition that targets Fusobacteria) to the human in combination one or more lymphoma treatments (e.g., chemotherapies, immunotherapies, radiation therapies, bone marrow transplants, and/or cell therapies such as a CAR-T cell therapies). The treatment is used to improve survival (e.g., cancer free survival) of the human having lymphoma.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-40. (canceled)

41. A method for treating a mammal having cancer, wherein said method comprises:
(a) identifying said mammal as having i-a) a reduced level of Akkermansia within the mammal's gut microbiome or (ii-a) a reduced level of Barnesiellaceae within the mammal's gut microbiome, and
(b) administering to said mammal a composition comprising (i-b) viable Akkermansia if (i-a) is identified or (ii-b) viable Barnesiellaceae if (ii-a) is identified, wherein said method is effective to improve survival of said mammal.

42. (canceled)

43. The method of claim 41, wherein said mammal is a human.

44. The method of claim 41, wherein said cancer is lymphoma.

45. The method of claim 41, wherein said method comprises identifying said mammal as having (i-a), and wherein said reduced level of Akkermansia is less than about 1% of the microbes present in the gut microbiome.

46. The method of claim 45, wherein said Akkermansia is selected from the group consisting of A. muciniphila, A. muciniphilia, A. glycaniphila, and combinations thereof.

47-52. (canceled)

53. The method of claim 41, wherein said method comprises identifying said mammal as having (ii-a), and wherein said reduced level of Barnesiellaceae is less than about 1% of the microbes present in the gut microbiome.

54. The method of claim 53, wherein said Barnesiellaceae is selected from the group consisting of B. viscericola, B. intestinihominis, B. sp904502265, B. excrementigallinarum, B. excrementipullorum, B. excrementavium, B. merdipullorum, B. merdigallinarum, and combinations thereof.

55-56. (canceled)

57. A method for treating a mammal having cancer, wherein said method comprises administering to said mammal a composition comprising viable Akkermansia or viable Barnesiellaceae, wherein said method is effective to improve survival of said mammal.

58. The method of claim 57, wherein said mammal is a human.

59. The method of claim 57, wherein said cancer is lymphoma.

60. The method of claim 57, wherein said composition comprises viable Akkermansia and viable Barnesiellaceae.

61. The method of claim 57, wherein said viable Akkermansia is selected from the group consisting of A. muciniphila, A. muciniphilia, and A. glycaniphila.

62-63. (canceled)

64. The method of claim 57, wherein said viable Barnesiellaceae is selected from the group consisting of B. viscericola, B. intestinihominis, B. sp904502265, B. excrementigallinarum, B. excrementipullorum, B. excrementavium, B. merdipullorum, and B. merdigallinarum.

65. (canceled)

66. The method of claim 57, wherein said method comprises administering to said mammal a lymphoma treatment.

67. A method for treating a mammal having cancer, wherein said method comprises:
(a) identifying said mammal as having an elevated level of Fusobacteria within the mammal's gut microbiome, and
(b) administering to said mammal a composition comprising an antibiotic to reduce the level of said Fusobacteria within said mammal, wherein said method is effective to improve survival of said mammal.

68. (canceled)

69. The method of claim 67, wherein said mammal is a human.

70. The method of claim 67, wherein said cancer is lymphoma.

71. The method of claim 67, wherein said elevated level of Fusobacteria is greater than about 0.1% of the microbes present in the gut microbiome.

72. The method of claim 67, wherein said Fusobacteria is selected from the group consisting of F. nucleatum, F. polymorphum, F. ulcerans, F. varium, F. mortiferum, F. perfoetens, F. animalis, F. vincentii, F. awasookii, F. periodonticum, F. russii, F. necrophorum, F. gonidiaformans, and combinations thereof.

73. The method of claim 67, wherein said method comprises administering to said mammal a lymphoma treatment.
